(19) United States
(12) Patent Application Publication
(10) Pub. No.: US 2015/0282705 A1
(43) Pub. Date: Oct. 8, 2015

(54) Method and System of Using Eye Tracking to Evaluate Subjects

(71) Applicant: Ofer Avital, Saint-Lazare (CA)

(72) Inventor: Ofer Avital, Saint-Lazare (CA)

(21) Appl. No.: 14/681,083

(22) Filed: Apr. 7, 2015

Related U.S. Application Data
(60) Provisional application No. 61/976,490, filed on Apr. 7, 2014.

(51) Int. Cl. A61B 3/113; A61B 5/055; A61B 5/16; A61B 5/0205

(52) U.S. Cl. CPC A61B 3/113; A61B 5/0205; A61B 5/055; A61B 5/168; A61B 5/02405

(57) ABSTRACT

A method and system of organizing salient, aversive, and neutral stimuli in a visual display, deliberately separated by space and/or time, to better reveal attractors and aversions for a test subject observing the display, whose reactions are tracked using eye and gaze tracking, as well as other optional body parameters, all captured as data. The data is analyzed using rules and algorithms to characterize or diagnose the subject based on single test results, or comparison to prior test results, and comparisons to norms. The method and system may be employed on stand-alone, off-the-shelf electronic devices, including smartphones, tablets, laptops, and desktops, as well as on higher-end computers systems, specialized equipment, and cloud-based set-ups, allowing mobile or setting-based usage. Optional sensor inputs may be utilized for the optional inputs described above.

### CROSS-REFERENCE TO RELATED APPLICATIONS

This application claims the benefit of U.S. Provisional Application No. 61/976,490, filed Apr. 7, 2014. The disclosure of the above application is incorporated herein by reference in its entirety.

### TECHNICAL FIELD

The invention disclosed herein generally relates to eye tracking (determination of gaze point or gaze angle) using a computer system. A test subject is shown visual stimuli, which trigger gaze patterns and other reactions, detected and stored as data that is analyzed and used to characterize and/or diagnose the subject. In particular, the invention provides an efficient method and system, employing particular design of the test-subject visual interface, particular stimuli selection, and rule-based and algorithmic data processing of acquired data. The method and system may advantageously be deployed on a diverse range of general purpose computing and communication devices without requiring additional hardware or software.

### BACKGROUND

All terms in this patent have the same meaning, whether capitalized or not.

### ABBREVIATIONS

GCVRDs: General-Purpose Computing and Video Recording Devices. These are general-purpose, usually mass-produced electronic computing and communication devices. GCVRDs preferably include, but are not limited to, mobile phones, tablets, netbooks, laptops, and desktops. They may also include higher-end servers (physical and virtual), server-client architectures, and cloud-based computing. They may also include a wearable computer with an optical head-mounted display

ETDCCs: Eye-Tracking-Relevant Diseases, Conditions, and Characteristics; those diseases, conditions, and Characteristics that may usefully be assessed via eye-tracking methods

ASD: Autism Spectrum Disorders

NT: Neurotypical (i.e. not autistic, and no other developmental delays)

### DEFINITIONS

“Test subject” or “subject” as described in this patent refers to a person, or other living thing, or a robot or other mechanical or computing apparatus, that may suitably be evaluated by the described method or system.

“Characteristic” means one of a very broad set of traits of a test subject, including but not limited to, disease, developmental stage, physiological state or mental state, or in a mechanical test subject, a malfunction, error, imprecision, or misprogramming. A user characteristic, will also include the following: a state of mind, attitude, ability, emotional state, preference, or other state of mind or body, of interest in either the health-related field, or in the fields of psychology, market research, web usability, robot testing and training, and other purposes well known and described in eye tracking prior art. Data collected may also be analyzed to assist in subtyping the test subject into subgroups of various physiological, psychological, and demographic groupings, for purposes in the aforementioned fields.

“Characterize” means to detect, predict, or otherwise infer one or more of a wide range of traits of a test subject.

“Computational Resources” means the finite, limited computational characteristics of a GCVRD or other computational device. The characteristics include various forms of memory/storage (e.g. RAM, long term memory, removable memory) processors (CPU, GPU, etc), displays, networked devices, and sometimes operating systems.

“Diagnose” means to screen, to diagnose, to monitor, to prognose, and to predict onset and the course of a disease, condition, characteristic of a subject,

Monitoring or tracking eye movements and detecting and analyzing a test subject's gaze point can be used in many different research, marketing, and health-related contexts. It can be used in the fields of psychology, market research, web usability, robot testing and training, and other purposes well known and described in eye tracking prior art. For example, data collected may be analyzed to assist in subtyping the test subject into subgroups of various physiological, psychological, reactional, demographic and other groupings, for purposes in the aforementioned fields. Eye tracking data can be an important information source in analysing the behaviour, consciousness, and neurological functions of a test subject. It is also used in the medical field to diagnose many diseases and conditions, ranging from autism to Alzheimer's disease.

Eye tracking research has traditionally employed high-resolution, high-end, dedicated, expensive cameras with enough resources to meet the needs of research. The corneal reflection eye tracking method is the most common, or one of the most common, methods used to study gaze performance. This method estimates the location of gaze with high accuracy (precision <1 visual degree, sampling rate 50 to 300 Hz) based on the reflection of near-infrared light from the cornea and the pupil. Gaze position is calculated by computer algorithms based on video recordings (showing the pupil and the near-infrared light reflections) collected by remote cameras placed in front of the observer. Eye tracking both improves measures obtainable with less advanced methods (for example, coding from video) and adds measures not available by other means, including fine-grained scanpath and fixation analyses (Ref: “Eye Tracking in Early Autism Research”, http://link.springer.com/article/10.1186/1866-1955-5-28/fulltext.html).

### SUMMARY OF THE INVENTION

This patent describes a method of organizing salient, aversive, mixed, and neutral stimuli in a visual display, deliberately separated by space and/or time, so as to better facilitate revelation of the attractors and aversions of a test subject observing the display. The subject's reactions are detected electronically, preferably by the standard video camera of a GCVRD, described later. System data inputs include the subject's eye position, movement and gaze pattern, as well as other optional body parameters (e.g. pupil response, facial expressions and symmetry, body and/or head movements, skin galvanic, EEG, etc., and others).

The resulting data, whether permanently recorded or not, is analyzed using special algorithms to diagnose and/or characterize ETDCCs and characteristics of the subject, based on comparisons of results in normative groups, results in groups with ETDCCs, derived clinical rules and detected characteristics, and/or even prior results from a given test subject. Additional data inputs may include data from various other body parameters from one or more input devices or sensors, as well as background data, for example, from a patient's history, physical exam and tests.

Further, this patent describes a method and apparatus for allowing useful performance of the method on off-the-shelf mobile devices and other off-the-shelf GCVRDs, rather than via obligatory use of the high-performance, expensive, bulky, eye tracking devices typically used in eye tracking applications. Such GCVRDs preferably include, but are not limited to, mobile phones, tablets, netbooks, laptops, and desktops. They may also include higher-end servers (physical and virtual), server-client architectures, and cloud-based computing. They may also include a wearable computer with an optical head-mounted display. They may also include computers which are affixed to advantageous stands, supports, frames, and patient support and fixation devices. Optional sensor inputs may be utilized for the optional inputs described above.

For utilization in the medical field, appropriate disease and medical conditions for the system to screen, diagnose, monitor, prognose, and predict, shall be called ETDCCs (Eye Tracking-Relevant Diseases, Conditions, and Characteristics). These include Autism Spectrum Disorder (ASD), stroke, cranial nerve palsies, drug intoxication, Traumatic Brain Injury (TBI), Alzheimer's Disease, Multiple Sclerosis, Parkinson's Disease, Amyotrophic Lateral Sclerosis (ALS) Fetal Alcohol Syndrome, ADHD, Tourette's syndrome, progressive supranuclear palsy (PSP), Huntington's disease (HD), brain tumors, impaired vision (including any or all of myopia, farsightedness, and other refractive or non-refractive visual defects), irregular blind spots, normative or irregular development and growth, and other conditions.

Note that throughout the patent, many references to ASD may be applied to other ETDCCs previously enumerated, and most references to neurotypical (NT) subjects can refer to an appropriate control group without the disease, condition, or characteristic.

Appropriate test subject characteristics to screen for using visual displays and eye tracking in consumer, marketing, and advertising testing, robot testing, and other domains are well described in their respective arts. Examples include preference for colour, shape, facial characteristics, product design, graphic design, and motion design.

### BRIEF DESCRIPTION OF THE DRAWINGS

FIG. 1 is prior art, a system described by Jones and Klin in U.S. Pat. No. 8,551,015, published in 2013 (“Patent J.1”): “a method for quantifying and mapping visual salience employable by the system shown in FIG. [2]”.

FIG. 2 is prior art, a system described in Patent J.1: “a system for quantifying and mapping visual salience”.

FIG. 2A is prior art, a system described in Patent PCT/US 2014/023644 (Klin et al): “systems and methods for detection of cognitive and developmental conditions”.

FIG. 3 is prior art, a system described by Ebisawa in U.S. Pat. No. 8,371,693, published in 2013 (“Patent E.1”); “a schematic configuration diagram showing an entire configuration of an autistic infant diagnosis apparatus according to a first embodiment of the present invention”.

FIG. 4 is prior art, a system described in Patent E.1: “a plan view showing placement of a color video camera for image capturing a face of a mother and an optical system for detecting a pupil position of the mother”.

FIGS. 5A-5F show layout configurations, part of the advantageous test subject user interface.

FIG. 6 shows, in block diagram form, one embodiment of the method in a system.

FIG. 7 shows an eye gazing at one stimulus on a display, then at another, with a wide gaze angle rotation required as movement between the two.

FIG. 8 is from the view of the device in FIG. 7, the video camera's view of the subject's eye looking at each stimulus, showing large, easily detected displacement between the two.

FIG. 9, analogous to FIG. 8, is a hypothetical illustration of output from prior art. From the view of the device in FIG. 7, the video camera's view of the subject's eye looking at each stimulus, showing small, difficult-to-detect displacement between the two.

FIGS. 10A-10B show deployment of the user interface method on a smartphone, tablet or other GCVRD.

FIG. 11 is a table-based organization of 7 invented clinical rules, which may optionally be stored in a database or other suitable format database in the system.

FIG. 12 shows test results from a hypothetical subject from three test episodes separated in time. Results are superimposed on a background showing normative standard development curves of risk for developing ASD based on test results at a given age. The three results from the subject show decreasing risk over time, possibly because of therapeutic interventions.

FIG. 13 is prior art, showing a wide distribution in the fixation time on eyes, from infant test subjects.

FIG. 14 shows a narrower distribution in the fixation time on eyes, from infant test subjects, as would be expected from utilization of our system.

FIG. 15 illustrates, in flow-diagram form, the pages that would be used in one embodiment of the system as an app on a handheld device.

FIGS. 16A-B illustrates, in flow-diagram form, operation of one embodiment of the system.

### DETAILED DESCRIPTION OF THE INVENTION

The following description is merely exemplary in nature. It will focus mainly on the diagnosis of ASD or other ETDCCs by describing use of the system, deployed on a common mobile device as an illustrative, and is in no way intended to limit the present teachings, application or uses.

One medical area of increasing interest is autism spectrum disorder (ASD). Early treatment can have a significant positive impact on the long-term outcome for children with ASD. Early treatment, however, generally relies on the age at which a diagnosis can be made, thus pushing early identification research into a category of high public health priority. Unfortunately, easily implemented methods for facilitating early screening and identification remain to be found (Ref: http://archpsyc.iamanetwork.com/article.aspx?articleid=210964).

Eye tracking technology holds promise as an objective method for characterizing the early features of autism because it can be implemented with individuals of virtually any age or functioning level. Historically, the bulk of eye tracking studies have been conducted with older children, adolescents, and adults with autism. In one of the first studies on this topic, Klin and colleagues showed that when watching a socially intense movie, adults with autism predominantly looked at the mouth region of the actors whereas typical subjects looked at the eye region. Bringing this effort into the childhood years, Jones and colleagues later showed that even 2-year-olds with autism spent more time fixating on the mouth region than the eyes during face viewing. They raised the provocative possibility that how social images are visually examined could be an early warning sign for autism.

Prior art references eye tracking systems for diagnosis of autism.

| | |
|---|---|
| US 2014/0213930 | (Mori) Patent Application Publication |
| U.S. Pat. No. 8,371,693 | (Ebisawa) Patent |
| EP 2,829,221 | (Mori) Patent Application “Asperger's |
| | Diagnosis . . . ” |
| PCT/US 2014/023644 | (Klin) Patent Application “Systems and |
| | Methods for Detection of Cognitive and |
| | Developmental Conditions” |

While eye tracking systems are utilized in research on patients with, or at risk for, various diseases including ASD, most are not yet practical enough to be used in most screening scenarios in clinical or mobile environments. Many or most primary care, hospital, and other institutions and environments that evaluate infants and children for health and development evaluation purposes (e.g. daycares, homes) could use such systems if they were practical. However, current systems, used in research centers, are bulky, expensive, usually immobile, and often require custom hardware such as special infra-red diodes, and do not necessarily fit into a non-research environment's workflow. They often take considerable time for test set-up and data capture. Many infants resist performing the test due to duration and ergonomic demands. Additionally, most current systems do not come equipped, or interface with, beneficial clinically-oriented analytics software clinical predictors, which can give a simple explanation of the clinical relevance of the test results. For example, a clinically useful risk stratification of disease probability could be that of a child's percentage probability to develop future ASD or other cognitive, developmental, or ophthalmic condition. Such algorithmic clinical analytics software in this field has also not been demonstrated to now in certain conditions. The limitations are similar for systems that could be used for evaluation of other many other ETDCCs.

Thus, there is a need for improved computer implementations of eye tracking and analysis systems for the evaluation of children at risk for ASD, and for patients with the other ETDCCs. This need also exists in any field utilizing eye tracking where testers wish specific and sensitive results of their subjects, especially in a portable manner and employing current devices that have limits to their computational resources and general specifications.

We have invented a method and system with a series of advantages, allowing the use of GCVRDs, to do what once required much more specialized, expensive, and less convenient equipment.

There exist other described methods and systems for using eye tracking to diagnose and assess autism spectrum disorder (ASD). Some of these methods and systems utilize a published preference of infants with ASD to preferably look to certain visual stimuli, in preference to other sorts of stimuli, and this preference being different to the preference of neurotypical (non-ASD affected) infants. One example of such a stimulus preference is a preference to look at the mouths of people in moving or still images (or at inanimate objects), instead of the more common behaviour of neurotypical infants which is to look preferentially at the eyes of people in such stimuli.

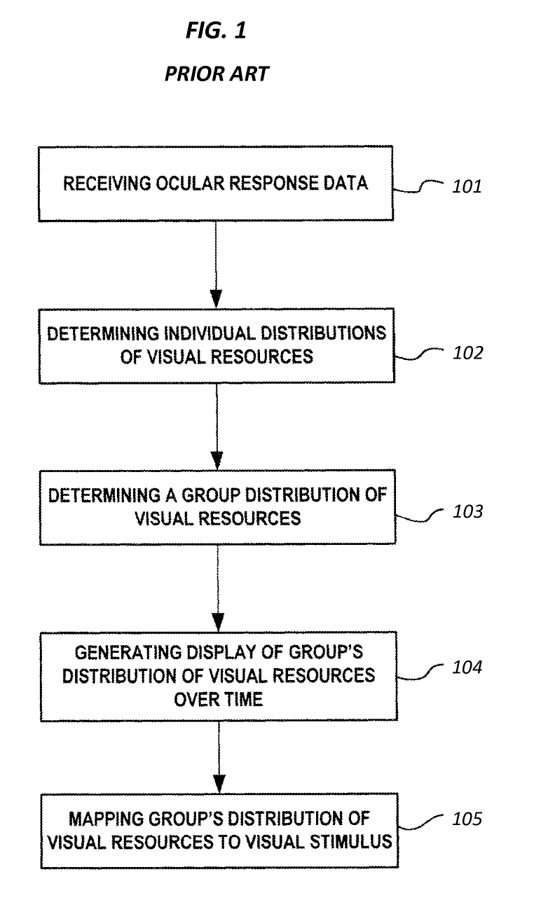

For example, U.S. Pat. No. 8,551,015 by Jones and Klin, published in 2013 (“Patent J.1”), describes a method FIG. 1 whereby infant subjects are shown a display with a single visual stimulus (e.g. a still image, video imagery, or interactive media). Eye tracking is performed on the subject watching the stimulus displayed on a 2-dimensional display. The subject's gaze pattern at the stimulus, over time, is preferably mapped into a 3-dimensional “attentional funnel”, mapping gaze movement in two dimensions, plus a third dimension showing time. The subject's gaze pattern, if autistic, will show a clear deviance, in such a 3-dimensional representation (called a “scanpath”), from the calculated bounds of a group of gaze patterns (scanpaths) derived from a group of neurotypical children or from children with certain other disorders. Neurotypical children's scanpaths are described as often converging towards eyes on a displayed face, where autistic children's scanpaths avoid eyes and tend to converge on other areas, such as mouth or objects. Many calculations, adjustments, and representations of the degree of deviance between groups of ASD and NT children, and between an individual with ASD and other groups are possible.

The method described in Patent J.1, while very advantageous as a research modality, has some clear limitations or disadvantages, as shall now be described.

First, it requires collecting, processing, and analyzing data on eye-movement/gaze from at least two different points in time, to generate a 3-dimensional “scanpath” demonstrating regions of gaze focus over a passage of time 104, 105. In fact, as described and illustrated in Patent J.1, it seems to typically require significantly more than two different points in time. This may require increased “Computational Resources” (including processing power, memory, storage requirements, graphics and display capabilities, and others) including more than that available on GCVRDs, as well as more time to perform the data capture. Longer time requirements also make data capture more difficult with restless young children and busy clinicians, making the method less practical in a screening setting.

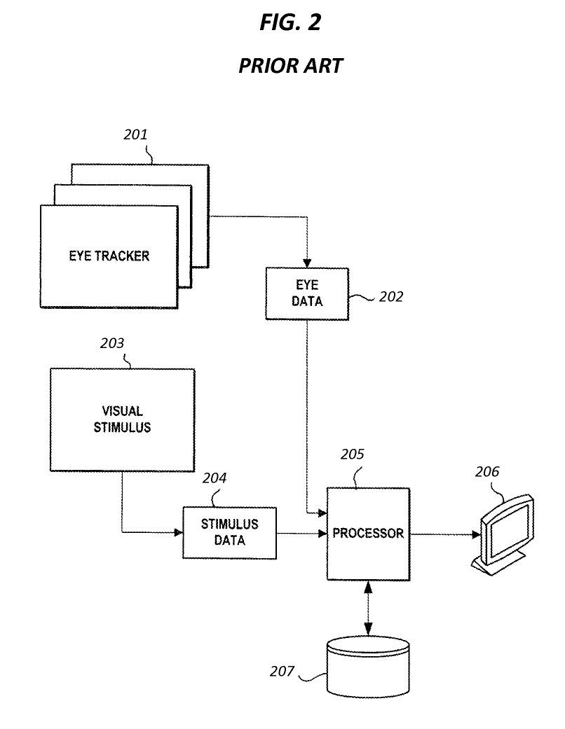

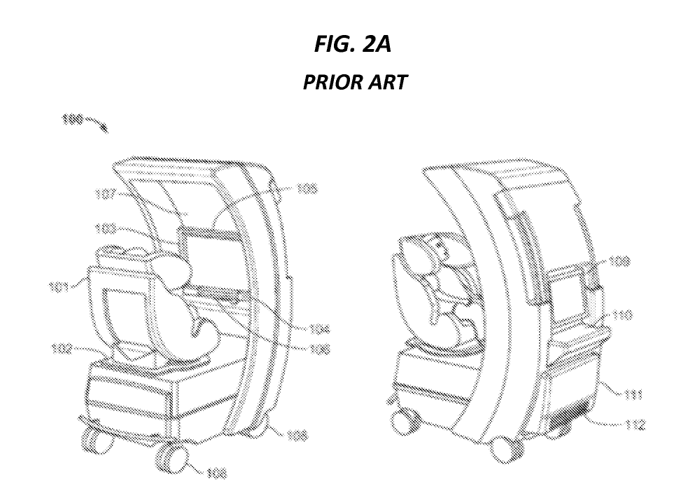

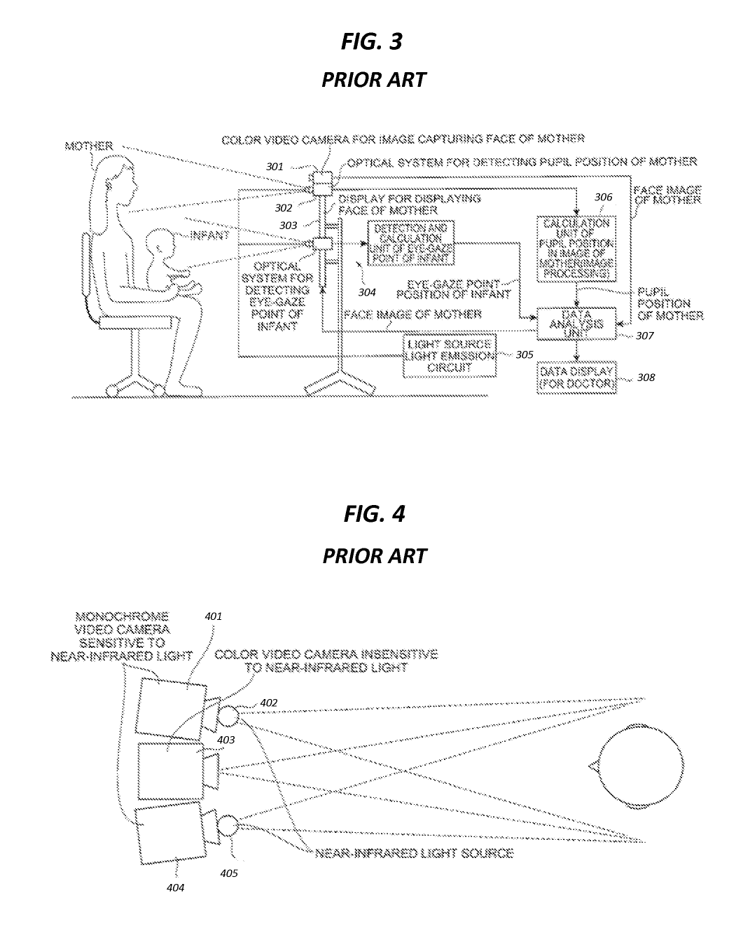

Second, it describes FIG. 2 requiring a separate “eye tracking device” or “eye tracker” 201 as is typically used in the art, listing as examples an infrared video-oculography device, or a binocular eye tracker. Another example of this sort of system in autism diagnosis is described and illustrated in U.S. Pat. No. 8,371,693 by Ebisawa (see 304, 401, 404), published in 2013 (“Patent E.1”). Eye tracking devices 201, as are typically used in the art, have their own data processors and algorithms which transform the visual input they receive, into “eye data” 202 indicative of ocular responses such as eye movements, direction, dilation, rotation, and/or gaze. Such data is then transmitted to a computer processor 205 in a computer, and it can then be stored and analyzed. Eye tracking devices, as are typically used in the art, are expensive, stand-alone, specialized and bulky devices, cannot be carried easily in a pocket, and are more suitable for a research setting than a clinical or screening setting. However, they are required for the particular method and system described in Patent J.1, Patent E.1, and that of many other patents, because the method and systems described require the generation and analysis of a massive amount of “eye data” involving extremely high-resolution accuracy, showing precisely where on a display a subject is looking at an exact time. This massive amount of data must then be compared to the even more massive norms of a control group to give meaning to the subject's “eye data”. This requires significant Computational Resources.

Third, it describes that the system further includes software for determining a subject's, or group of subjects′, distribution of “visual resources” at particular times 103, and from this, calculating an average value of “relative salience” and a “maximum salience”. This involves many calculations to compensate for where the external appearance of the eye appears to indicate that focus lies, as opposed to where the retina of the eye is actually focusing. This step is inherent in the method's requirement to resolve very fine degrees of gaze tracking, for example, that of an infant looking at a displayed face's eyes versus the face's mouth separated by only a few (angle) degrees of gaze. This requires yet additional algorithms, processing, and Computational Resources. However, an advantage of the high precision and varied parameters of measurement of this method is that it may be used as a possible confirmatory tool in a tertiary care center, in a situation where a screening method gave a result which was less than certain, and confirmation was desired.

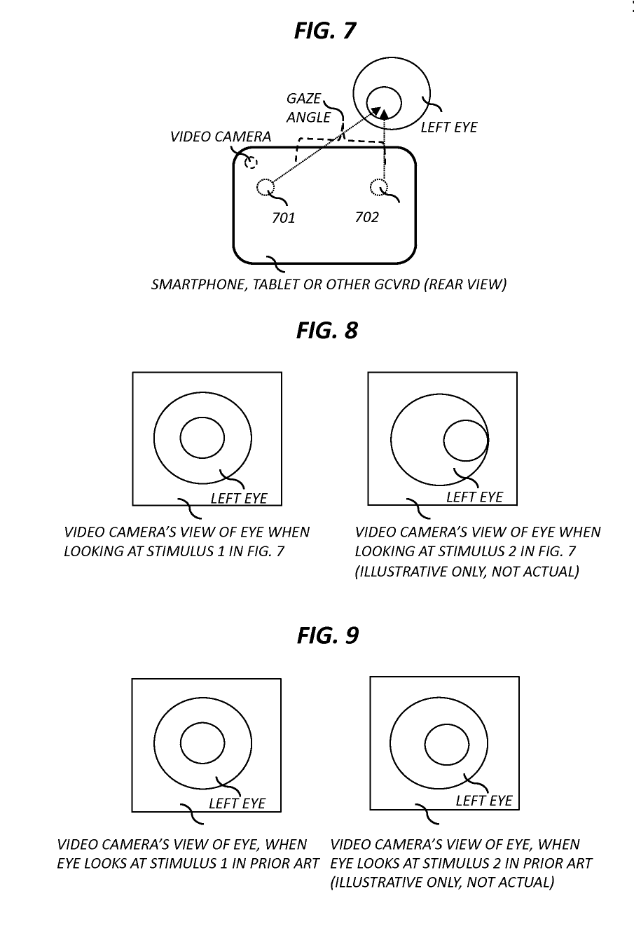

Fourth, there is no description for a preferred embodiment of a user-interface for the visual stimulus which could naturally increase the “gaze angle” FIG. 7 employed by a subject in looking towards, or away from, a visual trigger associated with a positive or negative diagnosis or result. If the subject were required to use a greater gaze angle to move its eyes from a noxious stimulus towards a salient stimulus, although still within the reasonable bounds of his abilities and condition, it is easier to perform capture of meaningful “eye data” without requiring equipment of higher technical specifications. As an example, a subject may need to move its eyes just a few degrees to shift between looking at a displayed face's eyes and mouth. If the displayed eyes and the mouth were deliberately more widely separated in space, a greater gaze angle change would result, and easier measurement of salience and aversion oculomotor movement. This could be done, for example, by placing a close-up of a pair of eyes in a region at far top of the screen 5C01, and of a mouth at the far bottom of the screen 5C02. Otherwise, to resolve such fine degrees of movement increases even further the required Computational Resources.

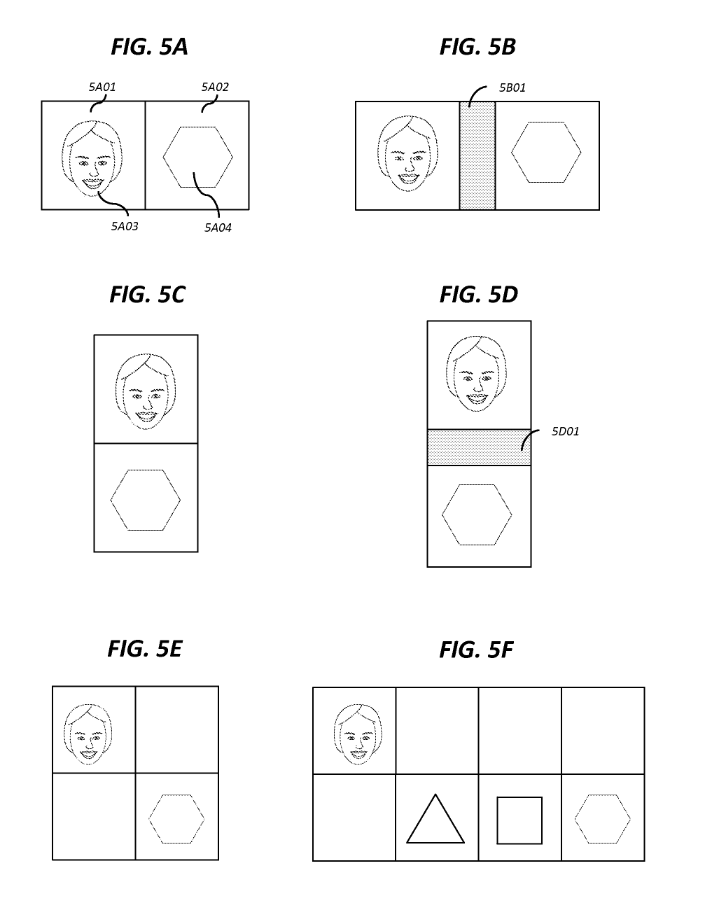

Fifth, in the method of Patent J.1, videos are analyzed for gaze over a very large number of possible fine points of attention represented as a very large amount of ocular response data 101. Our method, instead of typically analyzing very granular eye positions, movement, and therefore gaze patterns, has a “Resource Conserving Mode” of analyzing only for time spent gazing at a very reduced number of regions of interest that combine to occupy the entire viewing display. The “Resource Conserving Mode” utilizes precisely two to eight regions of interest for the testing user interface, where examples are illustrated in FIGS. 5A-5F. This decreases requirements for Computational Resources considerably, particularly since Computational demands grow enormously with increasing granularity and image resolution to process. As well, the user design reduces distracting elements in the visual display by filling distinct, major regions of the display with as much of an attractive or an aversive stimulus as possible. These regions by design are few in number, dividing the display into precisely two to eight regions, each region of equal size, except for “buffer” or “bridge” regions with no stimuli, and which are used to separate regions with stimuli; these regions may be smaller. In a “non-Resource Conserving Mode”, there is employed a far greater number of smaller regions of interest to analyze on the display, up to the limits of display resolution.

Sixth, while the method of Patent J.1 describes analysis of gaze patterns towards the visual stimulus' area(s) of “salience” (that “attract” gaze), there is no described deliberate employment of the opposite principle, namely the use of “aversive” stimuli. Autistic children are known to preferentially avoid looking at certain stimuli (e.g. biological motion, eyes, and more), where neurotypical children are not averse to looking at these, and in fact are usually attracted to them. The way to see if a child is averse to looking at a region of interest, as opposed to being primarily attracted to the adjoining region, is to design a visual stimulus and user interface to specifically differentiate between the two cases. One verifies if it differs in behaviour demonstrated towards an appropriate control or neutral visual stimulus, or if it differs from chance behaviour as can be readily calculated by methods described in the art.

Seventh, the method of Patent J.1 does not describe, nor does its method seem to allow for, a software-only solution that can be deployed on GCVRDs, already available in the pocket or the desktop of potential screeners today, without additional hardware. Significantly, such devices usually have far lower computing resources than the devices that the method of Patent J.1 and other patents describe, and have no separate high-performance gaze tracking device with its own dedicated computing resources. Additionally, GCVRDs will often have one or more of the following limiting characteristics: use of (1) normal-light, non-infrared, (2) monocular, video capture of (3) generic resolution, (4) expected utilization in varied lighting conditions, (5) expected utilization in a hand-held manner where shake or jitter of the image can reduce data capture, (6) with anti-shake stabilization functions built-in (or not) and (7) a zoom function that may not be as powerful as high-resolution zoom function in specialized gaze tracking devices (whether such zoom is mechanical or software driven).

In order to do so, this patent allows several methods to be employed.

A) Real-time processing: gaze analysis may be done in real time as the subject looks at the device.
B) Stored data: firstly, video recording is done and is then analyzed later, on the device or on a remote device. As a second option, in Resource-Conserving Mode or otherwise, data may be processed preferentially based on gaze on large regions of interest on the display.
C) Stored and batch processed: Another option is to process video in several batches during convenient time slots.

The increased speed in getting results from performing real-time analysis is an advantage, but probably not a critical one, given the relatively brief time required for processing recorded video, either in batches, or at once, as described in this method.

As well, in most screening situations, there will not be a need to see results immediately, as clinicians may want to look at other results and prepare before giving patients conclusions.

For batched or post-test analysis, many eye tracking analysis software components are available, ranging from open-source software (example: OpenCVS) to proprietary ones, or may be custom-written for this purpose.

For real-time processing, many video eye tracking analysis software components are available, ranging from OpenCVS to software from companies such as UMoove, and custom-made software may be made by a development team. Some of these software components can enhance the image-stabilization functions of GCVRDs, with further image stabilization techniques. Some of these software components enhance the light-adjustment capabilities of GCVRDs with further methods of adapting to various lighting conditions. Some of these utilize head-tracking, in addition to eye-tracking, to improve eye movement detection. Some of these can employ subpixel motion detection, by utilizing texture techniques instead of edge-detection techniques.

Expanding on the seventh point, one of the advantages of a software-only system that could deploy on GCVRDs with limited capabilities, is that such devices often already exist in the pocket or workflow of potential screeners, whom are already familiar and comfortable with their operation, and hence more likely to take up the use of a new screening system utilizing these devices. Clinicians could even delegate a portion of screening or of follow-up testing to parents or mobile workers to be advantageously performed in the home environment, on their own GCVRDs, with data optionally transmitted from home to clinician for actual analysis and evaluation. Whether utilized at a home, a clinic, or elsewhere, the overall system may also advantageously be used to compare progress or decline in health status over time, compared to a first baseline reading (see FIG. 12). This may also be useful to assess the effects or lack of effects of treatment. This utility would also be useful to assess intervention effects while conducting a clinical trial whose purpose was to assess the effectiveness of interventions for the ETDCC or a characteristic.

Eighth, the method of Patent J.1, while describing how unusual a scan-pattern is compared to a control group, does not describe an automatic process to translate that result into a simple clinical predictive output, such as the likelihood that an infant subject has ASD at the time of testing, or is on a developmental path of a certain risk to be diagnosed with ASD in the future. Such likelihood could be expressed as a percentage likelihood, relative risk, positive predictive value, negative predictive value, or as other commonly used clinical predictive or epidemiological measures regarding the disease or condition, whether in numerical, text, pseudo-text or other suitable output format.

Ninth, such likelihood of diagnosis can be automatically weighted or further adjusted based on other patient characteristics inputted into the system, such as many relevant factors from the patient history (e.g. family history or other known risks of ASD, patient IQ), the patient clinical exam (e.g. head lag upon lifting), or test results (e.g. blood, imaging, EEG, pupillary response, gene testing, proteomics, pathology). Clinical predictors that utilize more factors can give more specific results, which is desirable.

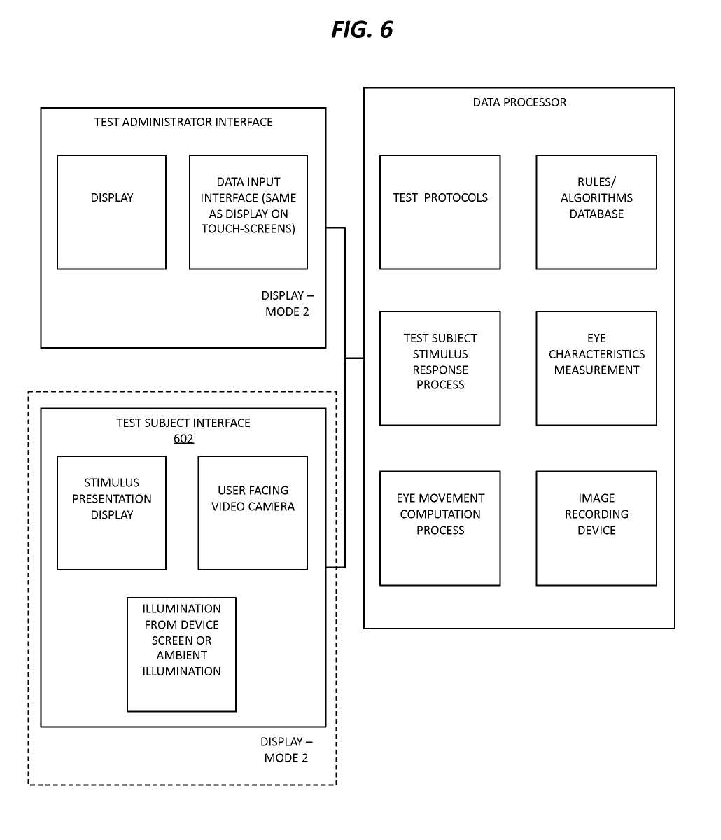

Our invention involves, in a first object, using improvements upon the visual paired preference paradigm in the Test Subject User Interface FIG. 6 602.

As described by Pierce, in “Eye Tracking in Early Autism Research” http://link.springer.com/article/10.1186/1866-1955-5-28/fulltext.html: In the paired preference paradigm, two visual displays that differ along one or more dimensions are presented side-by-side on a screen. This type of stimuli has a long tradition in developmental psychology. Frequently, the logic behind this approach is to be able to link looking time to a specific type of information. Thus, the fewer stimulus dimensions along which the two sides differ, the easier it will be to interpret the results. If processing of the information in question has established brain correlates, the results also will have implications at a neural level. Manipulating only one dimension at a time has historically been difficult to accomplish, often requiring follow-up experiments to exclude alternative explanations for results, for example why a subject looked longer at one display or stimulus.”

Our method, due to its many improvements on older methods of implementing this paradigm, has many advantages.

In another object, in our method we present a user interface on a display—see FIGS. 5A-5F where there are presented two or more visual stimuli 5A03 and 5A04, in a Left/Primary Region 5A01 and a Right/Paired Region 5A02, or in multiple regions as in 5F.

Additionally, there may be presented a neutral or bridge 5B01 5D01 section—see FIG. 5B and FIG. 5D; we can heighten the salience/aversion phenomenon by presenting a salient stimulus 5A04 in one region and an aversive stimulus 5A03 in another region at the same time. Or we can present either the salient (or the aversive) in a Primary region with neutral/absence in the Paired region at time 1, and then at time 2 display them in the opposite configuration to control for test subject preference to one side versus the other.

A common problem in utilization of the Paired preference paradigm is lack of control, between two different stimuli presented, for equivalence in their low-level visual properties, such as color, luminance, or amount of motion. Our method describes a significant improvement to this known limitation, via two solutions to this problem. Firstly, we can algorithmically pre-screen, and pre-tag, our stimuli according to these properties, and choose to simultaneously display only those which have similar low-level visual properties. Alternately, we can apply visual filters to stimuli to equalize these low-level parameters. The latter method advantageously allows use of more previously recorded stimuli, as well as new video stimuli that the tester can upload themselves, even via the generic video recording capability of the device they are using. For example, a mother may record a video stimulus of herself talking, and it may be filtered for low-level visual properties to be used in the system. Either of these solutions may be employed either real-time, or with offline processing, depending on system Computational Resources and convenience.

Results can be further simplified (and screening time decreased, and subject attention better maintained, increasing compliance and practicality) by showing very short videos composed of the salient/aversive options. For example, by using stimuli of approximately 1-4 seconds duration, these short duration stimuli advantageously reduce learning bias and boredom. Results of the output can be stored and analyzed for statistically significant gaze patterns, and advantageously can be analyzed for simple-to-calculate measures of interest, for example, time spent per region.

Such analysis and relatively simple outputs make for simpler subsequent calculations, and lower requirements for Computational Resources. For example, optionally the entire calculation can take place on a smartphone, without the need to transmit any results or data from the smartphone to a remote server for processing, although this latter could be an additionally employed step if desired for some reason, for example for backup storage, for interfacing with electronic medical records, for contribution to a group database for research or other purposes, for further processing, etc. More complex algorithms and calculations may be performed based on the hardware characteristics of the device employed.

Advantageously, our Primary and Paired regions can be adjusted to be not just placed Left-versus-Right as is typically done in the literature, but also in an Up-versus-Down arrangement FIG. 5C. This can be significant, because eye tracking can be affected by disease conditions or by background conditions, sometimes more in a particular direction (e.g. Left/Right but not Up/Down movements). For example, some neuropathies, caused by an infection, stroke, or other cause, can reduce the function of the cranial nerves that trigger oculomotor movements in very specific directions. In some cases, a subject's oculomotor range-of-motion may be decreased more in one direction than in another direction, either for one eye or for both eyes. In cases with more than one medical condition, the deficits can combine to give a mixed picture which requires further work to unpuzzle.

An object of our display would be to separate in space the two stimuli as far as practical, while still keeping them easily visible, to require the subject to perform a more significant gaze angle—see FIG. 7—oculomotor movement, which can therefore more easily be measured. The clear difference in ease of perception of a large gaze angle movement is demonstrated in FIG. 8 in the difference in what the video camera sees when the eye looks at stimulus 1 701 in FIG. 7, versus when the eye looks at stimulus 2 702. The difference in the apparent orientations of the two eye images in FIG. 8 is much easier to appreciate than the difference in FIG. 9, where the difference in FIG. 9 is often the degree of separation of relevant stimuli in prior art. The increased gaze angle forced in FIG. 8 also allows for measurement of the full range of motion of the eyes laterally, to allow for complimentary diagnosis of other disease states such as cranial nerve palsies. The range our method tests will seek to make eyes move to at least the four corners, including upper medial, lower medial, upper lateral, lower lateral, for each of the eyes. It is known in the art that having the eyes move in a path shaped like an “H” shape is convenient for testing these extremes of movement. The space dimension is generally the two-dimensional display of conventional off-the-shelf display screens, but may in another embodiment employ virtual, feigned, or actual, three dimensional placement of stimuli.

Advantageously, the eye data will record any asymmetry status of eye gaze upon test commencement, completion, and if system hardware permits, during gaze transitions. This is a further object of this invention, to help diagnose certain medical conditions, as well as to tease apart confounding elements in the case of having two medical conditions at once (for example ASD and a cranial nerve problem). In an alternative embodiment, further objects will be to optionally detect and record one or more of the following: (1) facial asymmetry, (2) static eye position resting asymmetry, (3) naso-labial fold asymmetry, and (4) smile asymmetry, all to assist in diagnosis. In an alternative embodiment, tracking saccades is also an object of this invention, to further aid in diagnosis and characterization. In an alternative embodiment, tracking blinks is also done.

In a paired presentation UI, one statistics solution is a simple time count of how much time is spent on Primary region 5A01 versus Paired region 5A02. The optional neutral “bridge” region 5B01 also has an amount of time spent with gaze passing over it. The object of recording this “bridge” time is to prevent attributing the time spent transitioning between the Primary and Paired region as time counted towards interest in the regions themselves, given that there is no focus in the region when gaze is diverting from it or heading towards it.

As another object of the method and system, the qualities and choices of the content selected to deploy within the regions of the UI are particular.

As to the content of these regions, we preferentially select for either stimuli with the highest salience, or the highest avoidance-reaction, or use a neutral/control/absent stimuli. An intervening “bridge” region between A and B will preferably have no content, or content selected to be neutral in terms of evoked reaction.

We would preferentially show stimuli with well described greatest differentiation between those with a ETDCC and those without, or use the system to discover new differentiators by iterative tests and comparisons. For example, in children with ASD, versus neurotypical children, (1) an aversion to eyes, and (2) an aversion to biological motion, versus to mechanical motion, are known amongst other differences. This list will grow to include other differentiating visual stimuli as they are discovered.

Another object of the invention is to look for avoidance, not just salience, by pairing stimuli with not simply competing stimuli in an adjacent region, but also against black screen or neutral backgrounds. Gaze time spent in a black or neutral background would indicate aversion and avoidance of the stimulus in the Primary region.

We can use multiple presentations of salient and aversive stimuli and neutral controls, mapping the combinations and the resulting gaze measurements to a data storage grid, representing the possibilities and likelihood of an associated diagnosis. The variations are optional dependent method enhancements.

Initial stimulus placement in either the Left (Primary) region or Right (Secondary) region can be in random or pseudo-random order. If a preference is noted for a particular image by the subject, that image can be shown more or less often in the future against competing stimuli, to help rank its relative interest in a qualitative or quantitative manner versus other stimuli. This method may be particularly interesting for consumer preference testing, robot testing, and other fields.

However, we also employ items whose rate of change in at least one aspect of appearance is “excessively complex” for the subject with ASD to process, and therefore may be irritating and aversive. In a display of a speaking person's face, the upper half of the face tends to be quite complex, carrying a lot of social information, but also simply a lot of movement, giving a possible explanation of why subjects with ASD avoid looking at the eye region.

In another implementation, we may display content that is “complex” for a subject with a different condition to characterize. For example, a visual maze or a word, or nonsense word, may be presented to a subject with possible ADHD or dyslexia, to see if their eye tracking and fixation patterns differ from a more typical pattern. The literature on attention-deficit disorder describes numerous visual challenges that may be employed.

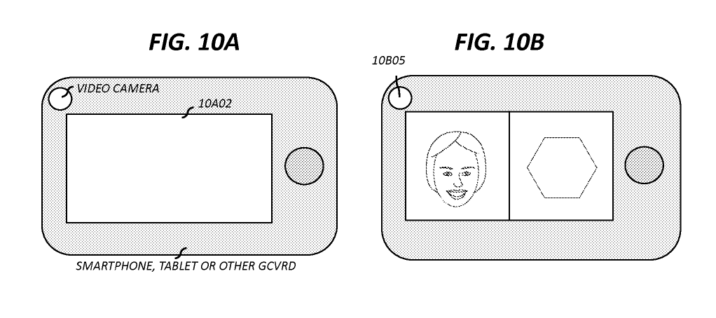

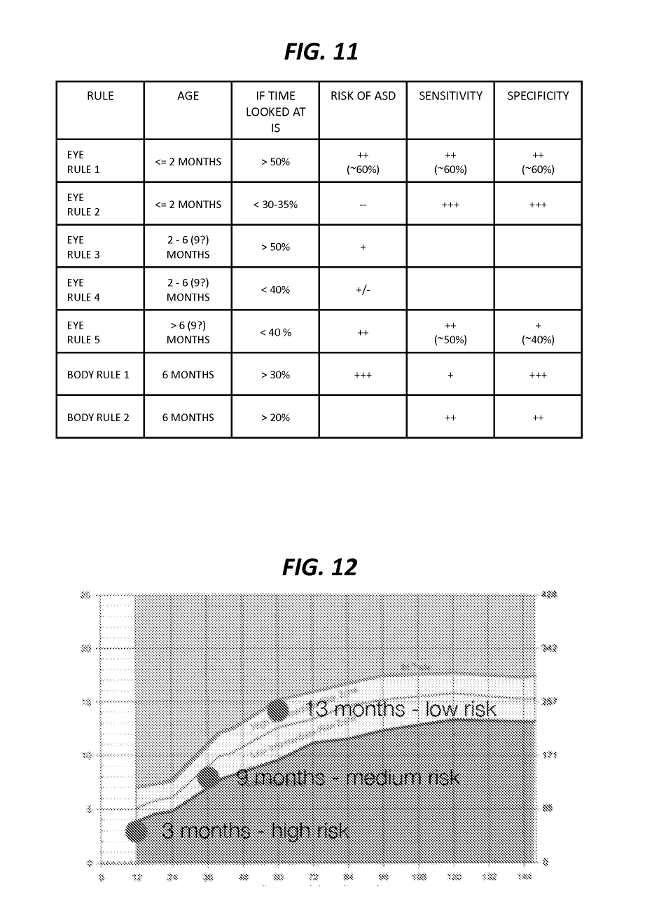

As another object, our system does not require the use of a separate “eye tracking device”. It is advantageously embodied in a software-only version, and utilizes simple video capture in the available resolution, sampling frequency, and light spectrum utilized by GCVRDs FIG. 10A or other previously described hardware utilized by the screening personnel. Mobile hardware typically has at least a basic video camera 10B05 as well as generic storage and processing capabilities typical of such devices for consumer and business use.

As another object, the system may be employed on GCVRDs with limited Computational Resources for processing and storage. This is accomplished via user interface design, design for usage very proximal to the test subject, design for decreased Computational Resource requirement, built-in camera usage, and utilization of general-purpose gaze tracking modules that may be (1) open source (e.g. OpenCVS), (2) licensable (e.g. from a commercial gaze-tracking software company such as Mirametrix or UMoove), or (3) custom-made.

Operating systems of such devices are typically iOS, Android, Linux, Unix, MAC OS, Windows, and other common systems. Preferably included is a basic display screen 10A02 for presenting stimuli to the test subject, although other stimuli substitutes, digital or otherwise, may be employed, for example, timed presentation of printed flashcards, or separate digital presentation screens which may be timing-synchronized to the GCVRDs. The software-only embodiment can be downloaded to a user's mobile device either as an app from an app store or via other appropriate means. To other GCVRDs it may be installed as is convenient. If appropriate to the workflow, the embodiment may communicate with remote computer systems via wi-fi, internet, or other wireless or wired system for data analysis, storage, consultations, connection with electronic medical records, research purposes, or other beneficial workflow purposes.

Another object of the invention is that the display is to be operated much closer to the subject than is used in many other eye tracking devices (typically within less than approximately a meter, and often within 1-2 feet). This permits the utilization of a display of significantly smaller dimensions, so that the display of a small mobile phone or tablet device will suffice. As well, the closer distance has the benefit of forcing yet greater gaze-angle utilization, and further decreasing resolution and hardware requirements.

In another object of the invention we describe a results page with relevant statistics. We can describe comparing patients to others in growth developmental curves FIG. 12, for example, percent time spent looking at eyes at a given age, and optionally trigger notices when, for example, 1 or 2 standard deviations of norms have been crossed from prior readings. The subject whose results are graphed in FIG. 12 is showing decreasing risk over time, possibly because of therapeutic interventions. Having this ability on a mobile device allows for mobile and remote diagnostics, which is a great advantage.

In another aspect of the invention, we describe the utilization of data, a series of predictive rules and algorithms for risk stratification and diagnosis.

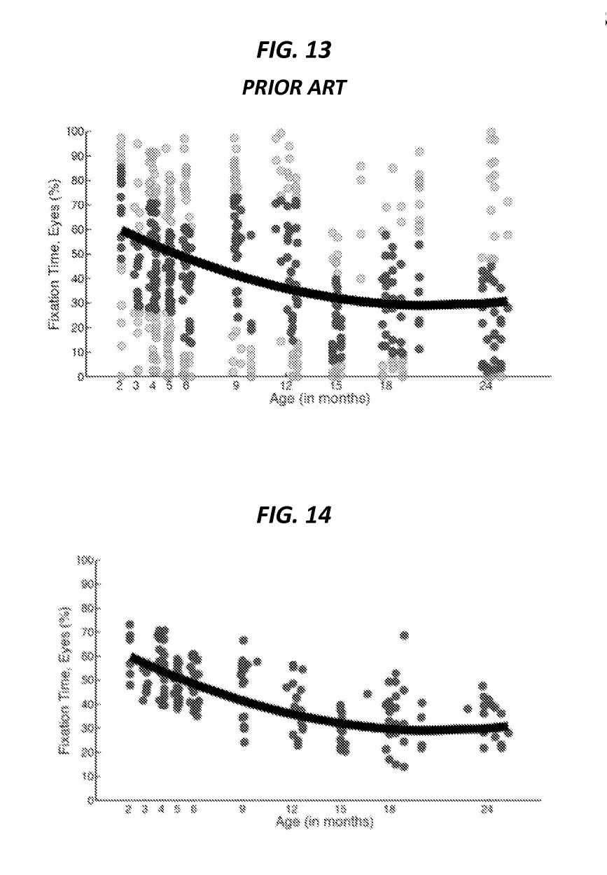

Firstly, due to our method, the data we receive from test subjects FIG. 14 may have reduced inherent variability and standard deviation compared to prior art FIG. 13. We would see a tighter zone of data, with less variance in graphs, for a subject's test results over time, and between individuals within one sort of condition grouping. This has advantages. It makes a diagnosis potentially more certain. It makes a diagnosis potentially possible with fewer testing events and shorter testing events. It makes a diagnosis potentially possible at an earlier age.

Secondly, as an example of a clinical rule using eye tracking in ASD, Pierce, in a 2010 research publication in Archives of General Psychiatry, states the following based on their data: “If a toddler spent over 69% of his or her time fixating on [repetitive] geometric patterns, then the positive predictive value for accurately diagnosing that toddler as having an ASD was 100%.

From examining various data, we have invented unique, new diagnostic clinical rules to diagnose specific conditions, for example, for ASD (TABLE T1).

Age ranges described in our rules may, in the future, be adjusted by approximately about 3-6 months, as more data comes in. As well, percentage time toddlers spent on eyes may change by about 10 or 20% in absolute terms as more data comes in.

++ refers to very high
+ refers to high
+/− refers to intermediate or mixed
− refers to low

Table showing 7 clinical rules.

TABLE T1

| | | | | | | RULE |
|---|---|---|---|---|---|---|
| | | | | | | SPECIFICITY |
| | | | IF TIME | | RULE | (i.e. % of time |
| | | | LOOKED | | SENSITIVITY | the rule gives |
| CLINICAL | | | AT | THEN RISK | (i.e. picks up | no false |
| RULE | AGE | STIMULUS | STIMULUSIS | OF ASD IS | % of cases) | positive) |
| RULE 1 | <=2 MONTHS | eye | >50% | ++ | ++ | ++ |
| | | | | (~60%) | (~60%) | (~60%) |
| RULE 2 | <=2 MONTHS | eye | <30-35% | −− | +++ | +++ |
| RULE 3 | 2-6 (9?) MONTHS | eye | >50% | + | | |
| RULE 4 | 2-6 (9?) MONTHS | eye | <40% | +/− | | |
| RULE 5 | >6 (9?) MONTHS | eye | <40% | ++ | ++ | + |
| | | | | | (~50%) | (~40%) |
| RULE 6 | 6 MONTHS | body | >30% | +++ | + | +++ |
| RULE 7 | 6 MONTHS | body | >20% | | ++ | ++ |

NB: Rate of ASD in population = 1 TO 2.5%. It is increased in boys.

Clinical Rule 8: if attention time towards eyes in a stimulus falls, from a prior month's reading [from between month 2 to month 12 of life], by at least 3%=very elevated risk of ASD (?˜90% specific) (˜85% sensitive).

Clinical Rule 9: if attention time towards body in a stimulus (versus towards eyes) does not fall from a prior month's reading [from between month 2 to month 12 of life] by at least 2-3%=very elevated risk of ASD (?˜90% specific) (˜85% sensitive).

Algorithm 1 can combine Rule 8× Rule 9 to increase sensitivity and specificity.

Clinical Rule 10: if child >=6 months looks at objects >20% of the time (in preference to eyes, mouth, and body)=++ risk, ++ specific, − sensitive.

Clinical Rule 11: if child >=6 months looks at body >30% of the time (in preference to eyes, mouth, and objects)=++ risk, ++ specific, − sensitive.

In another object of the invention, our predictive rules and outputs incorporate modifications in diagnoses/predictions from other patient characteristics, such as patient history, physical examination, and test results.

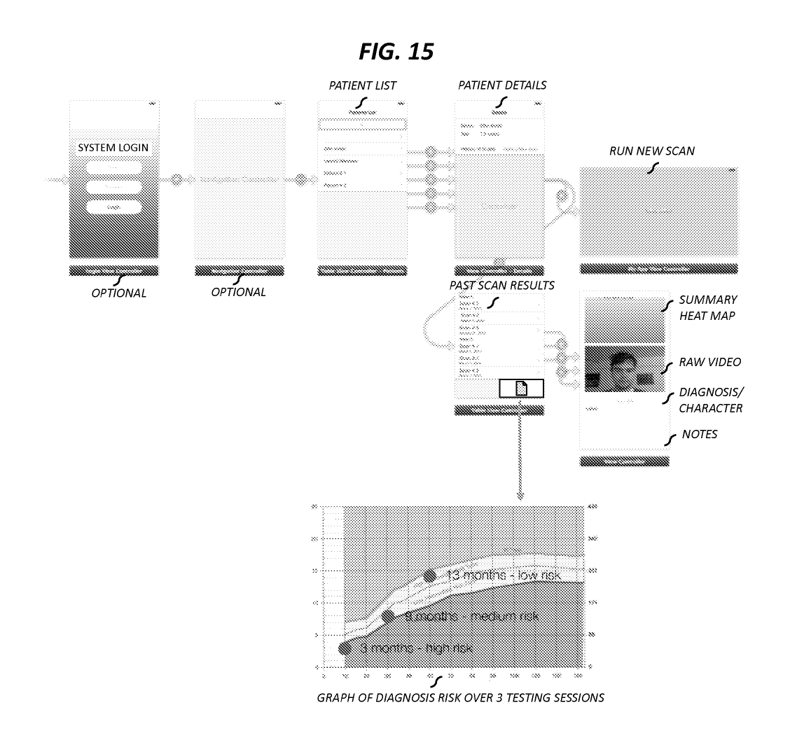

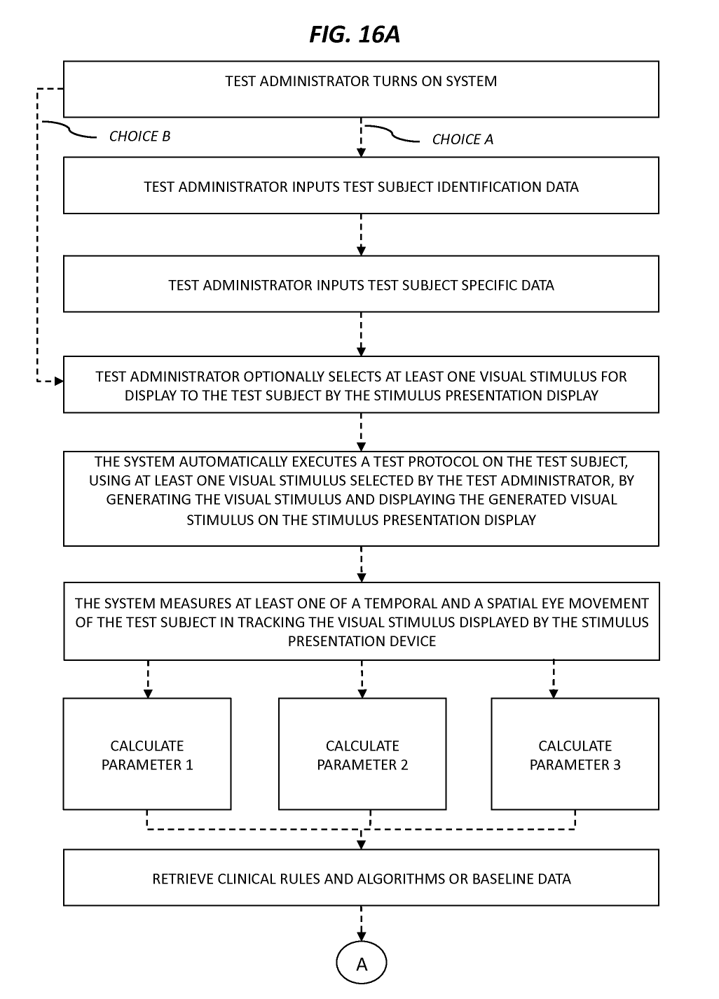

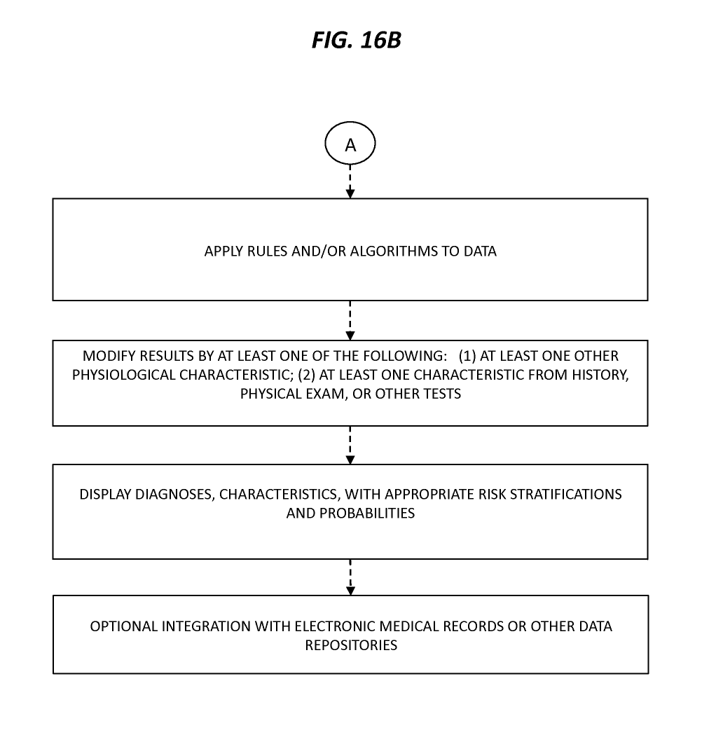

In another object of the invention, it has optional integration with a clinical workflow application FIG. 15. We illustrate one embodiment of the system as an app for a handheld GCVRD. It includes elements such as a patient list page, a patient details page, a start recording page, etc. The workflow is illustrated in FIG. 16A and FIG. 16B.

In another object of the invention, it has optional integration with a patient medical record storage system. The system may also advantageously be used to compare progress or decline in health status over time, compared to a first baseline reading. This may also be useful to assess the effects or lack of effects of treatment. This utility would also be useful to assess treatment effects while conducting a clinical trial whose purpose is to assess the effectiveness of treatments for the disease or condition.

What is claimed is:

1. A method of collecting data (that includes being advantageously employed utilizing a “mass-market” computing and/or communication device (GCVRDs—General-Purpose Computing and Video Recording Devices)), comprising:
use of a stimulus display interface design optimized for a particular GCVRD's display dimensions, capabilities, and data input capabilities (any or all of the three built-in or add-on);
use of a collection of stimuli of visual (and/or other sensory) content choices of aversive, attractive, neutral, and mixed stimuli, where such visual stimuli shall be displayed on a display module or system in at least one or more User Interface (UI) zones separated by space and/or time;
presentation of relevant stimuli on 1 or more (but particularly 2-8) UI display zones to a subject via the display methodology of the GCVRD (and/or attachments or networked devices) for example built-in screen, or projector, or via video output to another display or an e-paper display); and
detection of eye fixation or eye movement towards and away from a small number of UI zones (from 1-8) with optional bridge zones.

2. The method of claim 1, having UI zones and display content choices optimized, in part, to better elicit useful (for example more easily detectable or more easily performable) eye movements.

3. The method of claim 1, wherein a separation of stimuli in space and/or time is utilized to encourage the subject to employ a wide gaze angle, or a tall vertical gaze displacement.

4. The method of claim 1, wherein the method of placement of zones is optimized so gaze fixations and movements at one or more zones trigger one or more clinical rules to diagnose or characterize a subject.

5. The method of claim 1, where such UI design allows for positioning a recording camera, usually part of the GCVRD, advantageously closer to the subject's eye to better detect movements or change.

6. The method of claim 1, where such UI design allows for positioning the device closer to the subject so the device's opacity advantageously reduces distracting background elements.

7. The method of claim 1, including further data collection from at least one of visual, auditory, tactile, vestibular, electrical, and other sensory channels.

8. The method of claim 1, where stimuli to be displayed are controlled for low-level factors like color, brightness and speed of movement.

9. The method of claim 1, wherein a GCVRD's default built-in video camera is used to record subject eye movements and fixations.

10. The method of claim 1, where the data collection is performed at “good-enough” resolution to detect which of 1-8 zones the subject is looking at, towards, or away from the display at moments in time, requiring less computational resources.

11. The method of claim 1, where the data collection is performed at “high” resolution, limited by the actual or apparent display resolution of the GCVRD, employing more computational resources.

12. The method of claim 1, wherein another motion-detection method is used for gathering data on eye movement, such as ultrasound, electric field detection, magnetic detection, nuclear or magnetic resonance, either built-in or add-on to the GCVRD.

13. The method of claim 1, wherein a fixation target is used to trigger reflex eye movement of the subject.

14. The method of claim 1, wherein the eye movement of the subject is elicited without verbal instruction.

15. The method of any of the claims above, wherein eye asymmetry in position or movement is used to further inform a diagnosis.

16. The method of any of the claims above, wherein either or both of facial feature asymmetry and facial movement are further used to diagnose a subject, where data on such movement is gathered in a manner similar to the data on eye movement.

17. The method of any of the claims above, wherein in addition data is gathered and utilized from the modalities of Galvanic skin response, pulse, respiratory rate, heart rate, heart rate variability, EEG, muscle tension and are optionally used to diagnose a subject.

18. The method of any of the claims above, wherein the method is used for assessment, screening, monitoring, or to diagnose development or cognitive conditions or “characteristics” of a “subject”.

19. The method any of the claims above, wherein the method is used for assessment, screening, monitoring, or diagnosis of development of Autism.

20. The method of claim 19, (to diagnose Autism) where it is done by using a clinical rule (for example, from the table in the specification).

21. The method of claim 19, (to diagnose Autism) where it is done by comparing to similar results from controls and noting differences.

22. The method of claim 19, (to diagnose Autism) where it is done by comparing to similar results from subjects with known conditions.

23. The method of any of claims 1-17, wherein the method is used for assessment, screening, monitoring, or diagnosis of development of ADHD.

24. The method of any of claims 1-17, wherein the method is used for assessment, screening, monitoring, or diagnosis of development of another disease or characteristic.

25. A computer-readable medium containing instructions, which when executed on a general-purpose computer or communication device (GCVRD), perform the method of claim 1.

26. A computer-readable medium containing instructions, which when executed on a general-purpose computer or communication device (GCVRD), perform the method of any of claims 1-17.

27. A system that triggers and measures responses and collects data comprising:
i. a visual display controllable by a display signal;
ii. an imaging device adapted to image the face of a viewer of the visual display; and
iii. a processing card adapted to generate a display signal for causing said visual display to produce a screen pattern,
iv. said system characterized in a gaze-analysis module and optional diagnostic and/or a categorization module

28. A gaze tracking system according to claim 27, further comprising one or more reference illuminators, each being adapted to emit invisible light in order to produce a glint on a viewers eye, or to sub-threshold pixels to facilitate eye tracking by reflection of the eye
i. wherein the gaze tracking system is adapted to determine a gaze point of the viewers eye further based on the position of the glint.

29. A gaze tracking system according to claim 27, wherein said one or more reference illuminators are detachable.

30. A gaze tracking system according to claim 27, wherein the imaging device is synchronized with the visual display and/or a reference illuminator.

31. A gaze tracking system according to claim 27, wherein the imaging device is synchronized with the visual display and/or a reference illuminator.

32. A personal computer system (“GCVRD”) comprising a system of collecting data according to claim 25 or claim 26.

33. A system of collecting data, comprising at least a GCVRD, using the method of any of claims 1-24.
